(19) United States
(12) Patent Application Publication
(10) Pub. No.: US 2019/0353805 A1
(43) Pub. Date: Nov. 21, 2019

(54) DIGITAL X-RAY DETECTOR HAVING POLYMERIC SUBSTRATE

(71) Applicant: General Electric Company, Schenectady, NY (US)

(72) Inventors: Nicholas Ryan Konkle, Sussex, WI (US); Daniel Robert Lochner, West Allis, WI (US); Douglas Albagli, Clifton Park, NY (US); Joseph John Kulak, Milwaukee, WI (US); William Andrew Hennessy, Troy, NY (US); Habib Vafi, Brookfield, WI (US)

(21) Appl. No.: 15/984,953

(22) Filed: May 21, 2018

(51) Int. Cl. G01T 1/24; H01L 27/146; A61B 6/00

(52) U.S. Cl. CPC G01T 1/247; A61B 6/4405; H01L 27/14676; G01T 1/243

(57) ABSTRACT

A digital X-ray detector is provided. The digital X-ray detector includes a polymeric substrate. The digital X-ray detector also include a detector array configured to generate image data based on incident X-ray radiation disposed on the polymeric substrate, wherein the polymeric substrate extends beyond an edge of the detector array. The digital X-ray detector further includes scan electronics and readout electronics configured to acquire image data from the detector array, wherein the scan electronics, the readout electronics, or both the scan electronics and the readout electronics are directly disposed on the polymeric substrate.

### BACKGROUND

The subject matter disclosed herein relates to X-ray imaging systems and more particularly to a portable X-ray detector having a polymeric substrate.

A number of radiological imaging systems of various designs are known and are presently in use. Such systems generally are based upon generation of X-rays that are directed toward a subject of interest. The X-rays traverse the subject and impact a film or a digital detector. In medical diagnostic contexts, for example, such systems may be used to visualize internal tissues and diagnose patient ailments. In other contexts, parts, baggage, parcels, and other subjects may be imaged to assess their contents and for other purposes.

Increasingly, such X-ray systems use digital circuitry, such as solid-state detectors, for detecting the X-rays, which are attenuated, scattered or absorbed by the intervening structures of the subject. Solid-state detectors may generate electrical signals indicative of the intensities of received X-rays. These signals, in turn, may be acquired and processed to reconstruct images of the subject of interest.

As digital X-ray imaging systems have become increasingly widespread, digital X-ray detectors have become more portable for even greater versatility. Typically, a 2D flat panel array of silicon photo-detectors is fabricated on (e.g., via lithography) on a thin fragile glass substrate to form the X-ray detector panel or imaging panel. The panel is typically heavy and brittle. In addition, the detector includes additional components (e.g., panel support) to protect and limit the flexibility of the imaging panel. This results in a thicker and heavier detector. However, it is desirable to have a thin and lightweight X-ray detector.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In accordance with a first embodiment, a digital X-ray detector is provided. The digital X-ray detector includes a polymeric substrate. The digital X-ray detector also include a detector array configured to generate image data based on incident X-ray radiation disposed on the polymeric substrate, wherein the polymeric substrate extends beyond an edge of the detector array. The digital X-ray detector further includes scan electronics and readout electronics configured to acquire image data from the detector array, wherein the scan electronics, the readout electronics, or both the scan electronics and the readout electronics are directly disposed on the polymeric substrate.

In accordance with a second embodiment, a digital X-ray detector is provided. The digital X-ray detector includes a polymeric substrate. The digital X-ray detector also include a detector array configured to generate image data based on incident X-ray radiation disposed on the flexible polyimide substrate. The detector array includes a scintillator configured to convert the incident radiation into lower energy optical photons, a semi-hermetic coating or hermetic conductive coating is disposed on a side of the polymeric substrate opposite the scintillator and a flexible seal is disposed over the scintillator. The flexible seal is bonded to the polymeric substrate at a location beyond an edge of the detector array to semi-hermetically or hermetically seal the scintillator so that the edge of the polymeric substrate is also semi-hermetically or hermetically sealed.

In accordance with a third embodiment, a method for manufacturing a digital X-ray detector is provided. The method includes depositing a detector array configured to generate image data based on incident X-ray radiation on a polymeric substrate disposed on a glass substrate wherein the polymeric substrate extends beyond an edge of the detector array and then bends. The method also includes removing the glass substrate from the polymeric substrate. The method further includes directly disposing scan electronics, readout electronics, or both the scan electronics and the readout electronics on the polymeric substrate on the bend or after the bend of the polymeric substrate, wherein the scan electronics and the readout electronics are configured to acquire the image data from the detector array.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical overview of a digital X-ray imaging system in which the present technique may be applied;

FIG. 2 is a diagrammatical representation of components in a detector of the system of FIG. 1;

FIG. 3 is a diagrammatical representation of components of a detection subsystem (e.g., configured for front side irradiation) in a detector of the system of FIG. 1;

FIG. 4 is a diagrammatical representation of components of a detection subsystem (e.g., configured for back side irradiation) in a detector of the system of FIG. 1;

FIG. 5 is a diagrammatical representation of components of a detection subsystem (e.g., having carbon fiber or plastic substrate) in a detector of the system of FIG. 1;

FIG. 6 is a diagrammatical representation of components of a detection subsystem (e.g., having a detector array sealed) in a detector of the system of FIG. 1;

FIG. 7 is a diagrammatical representation of components of a detection subsystem (e.g., having a detector array sealed via a seal at the edge of a substrate) in a detector of the system of FIG. 1;

FIG. 8 is a diagrammatical representation of a portion of a detection subsystem (e.g., configured for front side and back side irradiation at the same time) in a detector of the system of FIG. 1;

FIG. 9 is a bottom view of an imager disposed on a flexible substrate; and

FIG. 10 is an embodiment of a method for manufacturing a detector subsystem of a detector of the system of FIG. 1.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The following embodiments describe a portable digital X-ray detector that includes a detector subsystem with a polymeric (e.g., flexible polyimide) substrate or panel. The polymeric substrate along with other flexible components of the detector subsystem (e.g., scintillator) makes the detector subsystem flexible and more rugged. The substrate may extend (and bend) beyond an edge of a detector array of the detector subsystem. In certain embodiments, scan and readout electronics may be disposed on the polymeric substrate at or beyond the bend. In certain embodiments, the scan and readout electronics disposed on the polymeric substrate may be located on the opposite side of detector subsystem (e.g., behind the imager) from where incident X-ray radiation is received to minimize exposure of the scan and readout electronics to radiation. The utilization of the polymeric substrate may also enable the imager to extend closer to the edge of the detector. In certain embodiments, components of a plurality of imager modules may be disposed in a tiled arrangement on the polymeric substrate to enable multiplexed readout of the image data.

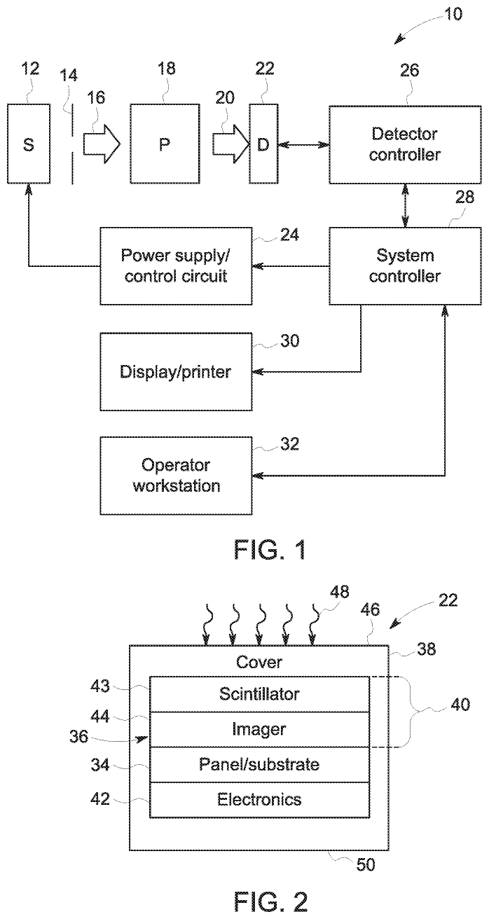

Turning now to the drawings, FIG. 1 illustrates diagrammatically an imaging system 10 for acquiring and processing discrete pixel image data. In the illustrated embodiment, system 10 is a digital X-ray system designed both to acquire original image data and to process the image data for display in accordance with the present technique. The imaging system 10 may be a stationary system disposed in a fixed X-ray imaging room or a mobile X-ray system. In the embodiment illustrated in FIG. 1, imaging system 10 includes a source of X-ray radiation 12 positioned adjacent to a collimator 14. Collimator 14 permits a stream of radiation 16 to pass into a region in which a subject, such as a human patient 18 is positioned. A portion of the radiation 20 passes through or around the subject and impacts a digital X-ray detector, represented generally at reference numeral 22. The detector 22 is portable. In certain embodiments, the detector 22 may convert the X-ray photons incident on its surface to lower energy photons, and subsequently to electric signals, which are acquired and processed to reconstruct an image of the features within the subject. In other embodiments, such as in a direct conversion implementation, the incident radiation itself may be measured without an intermediary conversion process.

Source 12 is controlled by a power supply/control circuit 24 which furnishes both power and control signals for examination sequences. Moreover, detector 22 is coupled to a detector controller 26 which commands acquisition of the signals generated in the detector 22. Detector controller 26 may also execute various signal processing and filtration functions, such as for initial adjustment of dynamic ranges, interleaving of digital image data, and so forth. Both power supply/control circuit 24 and detector controller 26 are responsive to signals from a system controller 28. In general, system controller 28 commands operation of the imaging system to execute examination protocols and to process acquired image data. In the present context, system controller 28 also includes signal processing circuitry, typically based upon a general purpose or application-specific digital computer; and associated manufactures, such as optical memory devices, magnetic memory devices, or solid-state memory devices, for storing programs and routines executed by a processor of the computer to carry out various functionalities (e.g., gain calibration and gain correction), as well as for storing configuration parameters and image data; interface protocols; and so forth. In one embodiment, a general or special purpose computer system may be provided with hardware, circuitry, firmware, and/or software for performing the functions attributed to one or more of the power supply/control circuit 24, the detector controller 26, and/or the system controller 28 as discussed herein.

In the embodiment illustrated in FIG. 1, system controller 28 is linked to at least one output device, such as a display or printer as indicated at reference numeral 30. The output device may include standard or special purpose computer monitors and associated processing circuitry. One or more operator workstations 32 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

Referring now to FIG. 2, a diagrammatical representation of a digital X-ray detector 22 having a flexible polymeric panel or substrate 34 (e.g., polyimide) is illustrated. The detector 22 includes a detection subsystem 36 disposed within a shell assembly or enclosure 38. The detection subsystem 36 includes digital detector array 40, a flexible polymeric substrate 34, and electronics 42. As depicted, the digital detector array 40 includes a scintillator 43 and an imager 44 (e.g., light imager having photodetector layer). In certain embodiments, the scintillator 43 may be directly deposited on the imager 44. In certain embodiments, the scintillator 43 may also be flexible. As noted above, in certain embodiments, the detector 22 may not include a scintillator but instead may measure incident radiation itself without an intermediary conversion process. The polymeric substrate 34 supports the detector array 40 and the electronics 42. In certain embodiments, the polymeric substrate 34 may be bonded on a rigid support (e.g., flat or curved support). The rigid support provides a reliable shape that enables the attachment of the rest of the detector components. The rigid support may be made of aluminum, magnesium alloys, carbon fiber, engineering plastics (e.g., polycarbonate or other material), or a combination thereof. In certain embodiments, the detection subsystem 36 may include additional layers for electromagnetic interference (EMI) shielding, reflection of optical photons towards the scintillator, etc. The flexible panel or substrate 34 enables the detector 22 to lighter and more rugged. Also, the flexible panel or substrate 34 enables other features for the detector 22 as described in greater detail below.

As noted above, the polymeric substrate 34 may be polyimide. The polyimide substrate may include the following thermal properties. The polyimide substrate may include a coefficient of thermal expansion ranging from approximately 25 to 30 ppm/° C. (at a test condition of approximately 100 to 200° C.). The polyimide substrate may include heat shrinkage ranging from −0.022 to −0.030% (at a test condition of 150° C. for 30 minutes).

As to other components of the digital detector 22, the electronics 42 control the operation of the detector 22. In particular, electronics 42 enable the acquisition of image data from the digital detector array 40. The electronics 42 may include scan and readout electronics including circuit boards, data modules, scanning modules, and other circuitry.

The scintillator 43 converting incident X-rays to visible light. The scintillator 43, which may be fabricated from cesium iodide (CsI), gadolinium oxysulfide (GOS), or other scintillating materials, is designed to emit light proportional to the energy and the amount of the X-rays absorbed. As such, light emissions will be higher in those regions of the scintillator layer where either more X-rays were received or the energy level of the received X-rays was higher. Since the composition of the subject will attenuate the X-rays projected by the X-ray source to varying degrees, the energy level and the amount of the X-rays impinging upon the scintillator 43 will not be uniform across the scintillator layer. This variation in light emission will be used to generate contrast in the reconstructed image.

The light emitted by the scintillator 43 is detected by a photosensitive layer on the imager 44. The photosensitive layer includes an array of photosensitive elements or detector elements to store an electrical charge in proportion to the quantity of incident light absorbed by the respective detector elements. Generally, each detector element has a light sensitive region and a region including electronics to control the storage and output of electrical charge from that detector element. The light sensitive region may be composed of a photodiode, which absorbs light and subsequently creates and stores electronic charge. After exposure, the electrical charge in each detector element is read out using logic-controlled electronics 42. In certain embodiments, due to the flexibility of the substrate 34, the detector 22 may include more than one scintillator and/or more than one imager.

Each detector element is generally controlled using a transistor-based switch. In this regard, the source of the transistor is connected to the photodiode, the drain of the transistor is connected to a readout line, and the gate of the transistor is connected to a scan control interface disposed on the electronics 42 in the detector 22. When negative voltage is applied to the gate, the switch is driven to an OFF state, thereby preventing conduction between the source and the drain. Conversely, when a positive voltage is applied to the gate, the switch is turned ON, thereby allowing the photodiode to be recharged, with the amount of charge being a function of the diode depletion as an indication of incident energy, which is detected on the readout line. Each detector element of the detector array 40 is constructed with a respective transistor (e.g., a thin-film transistor).

Specifically during exposure to X-rays, negative voltage is applied to all gate lines resulting in all the transistor switches being driven to or placed in an OFF state. As a result, any charge depletion experienced during exposure reduces the charge of each detector element. During read out, positive voltage is sequentially applied to each gate line. That is, the detector is an X-Y matrix of detector elements and all of the gates of the transistors in a line are connected together so that turning ON one gate line simultaneously reads out all the detector elements in that line. A multiplexer may also be used to support read out of the detector elements in a faster fashion. The output of each detector element is then input to an output circuit (e.g., a digitizer) that digitizes the acquired signals for subsequent image reconstruction on a per pixel basis. In a typical reconstruction, each pixel of the reconstructed image corresponds to a single detector element of the digital detector array 40.

The enclosure 38 protects the detector components from damage when exposed to an external load or an impact. The enclosure 38 includes a front side 46 to receive radiation 48 (e.g., during front side irradiation). In certain embodiments, the detector 22 may also be may receive radiation on the back side 50 (e.g., during back side irradiation). In certain embodiments, the detector 22 may be utilized for both front side and back side irradiation. The enclosure 38 may be formed of materials such as a metal, a metal alloy, a plastic, a composite material, or a combination of the above. In certain embodiments, the material has low X-ray attenuation characteristics. In one embodiment, the enclosure 38 may be formed of a lightweight, durable composite material such as a carbon fiber reinforced plastic material, carbon reinforced plastic material in combination with foam cores, or a graphite fiber-epoxy composite. Some embodiments may include one or more material compositions having a non-conductive matrix with conductive elements disposed therein, and may provide electromagnetic interference shielding to protect the internal components of the detector 22 from external electronic noise. Additionally, the enclosure 38 may be designed to be substantially rigid with minimal deflection when subjected to an external load.

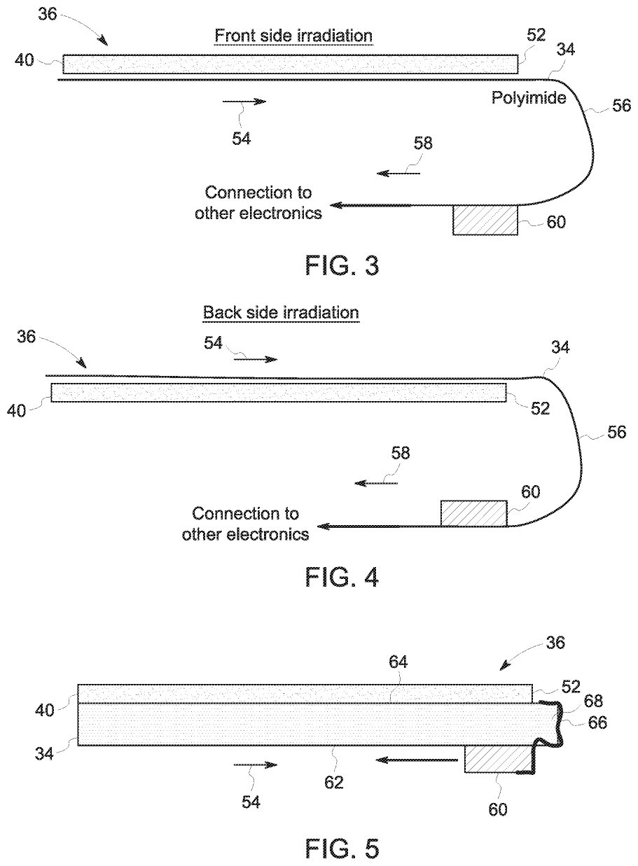

FIG. 3 is a diagrammatical representation of components of the detection subsystem 36 (e.g., configured for front side irradiation) in the detector 22 of the system 10 of FIG. 1. As depicted, the detector array 40 is disposed on the polymeric substrate 34. The polymeric substrate 34 extends beyond an edge 52 of the detector array 40 in a first direction 54. In certain embodiments, the polymeric substrate 34 may extend beyond multiple edges of the detector array 40. The portion of the polymeric substrate 34 extending beyond the edge of the detector array 40 includes a bend 56 that enables the substrate 34 to extend in a second direction 58 opposite the first direction 54 after the bend 56. As depicted, scan and readout electronics (represented by die or chip 60) may be disposed after the bend 56 on the polymeric substrate 34 and subsequently be connected with other electronics. The die or chip 60 is disposed behind the detector array 40 (and a portion of the substrate 34) to minimize exposure to incident radiation (since the radiation first impacts the detector array 40 and then the substrate 34). This also enables the detector array 40 to be extended further toward the edges of the detector 22. In certain embodiments, the scan and readout electronics 60 may be disposed on the substrate 34 on the bend 56. In certain embodiments, the digital control circuitry and power supplies may also be disposed on the substrate 34 on or after the bend 56. The die or chip 60 is coupled to the detector array 40 via metal traces (not shown) disposed on the substrate. In certain embodiment, the traces may be additively manufactured (e.g., 3D printed) on the substrate 34. In certain embodiments, the substrate 34 may be transparent to enable alignment of the imager modules and/or scan and readout electronics on the substrate. In certain embodiments, the substrate 34 may be black to block light to the electronics and/or imager. In other embodiments, the substrate 34 may be white.

FIG. 4 is a diagrammatical representation of components of the detection subsystem 36 (e.g., configured for back side irradiation) in the detector 22 of the system 10 of FIG. 1. As depicted, the detector array 40 is disposed on the polymeric substrate 34. The polymeric substrate 34 extends beyond the edge 52 of the detector array 40 in the first direction 54. In certain embodiments, the polymeric substrate 34 may extend beyond multiple edges of the detector array 40. The portion of the polymeric substrate 34 extending beyond the edge of the detector array 40 includes the bend 56 that enables the substrate 34 to extend in the second direction 58 opposite the first direction 54 after the bend 56. As depicted, scan and readout electronics (represented by die or chip 60) may be disposed after the bend 56 on the polymeric substrate 34 and subsequently be connected with other electronics. The die or chip 60 is disposed behind the detector array 40 (and a portion of the substrate 34) to minimize exposure to incident radiation (since the radiation first impacts the substrate 34 and then the detector array 40). This also enables the detector array 40 to be extended further toward the edges of the detector 22. In certain embodiments, the scan and readout electronics 60 may be disposed on the substrate 34 on the bend 56. In certain embodiments, the digital control circuitry and power supplies may also be disposed on the substrate 34 on or after the bend 56. The die or chip 60 is coupled to the detector array 40 via metal traces (not shown) disposed on the substrate. In certain embodiment, the traces may be additively manufactured (e.g., 3D printed) on the substrate 34. In certain embodiments, the substrate 34 may be transparent to enable alignment of the imager modules and/or scan and readout electronics on the substrate. In certain embodiments, the substrate 34 may be black to block light to the electronics and/or imager. In other embodiments, the substrate 34 may be white.

FIG. 5 is a diagrammatical representation of components of a detection subsystem 36 (e.g., having a carbon fiber or plastic substrate) in the detector 22 of the system 10 of FIG. 1. As depicted, the detector array 40 is disposed on a carbon fiber or plastic substrate 34. The substrate 34 extends beyond the edge 52 of the detector array 40 in the first direction 54. In certain embodiments, the substrate 34 may extend beyond multiple edges of the detector array 40. As depicted, scan and readout electronics (represented by die or chip 60) may be directly disposed on a side 62 of the substrate 34 opposite a side 64 where the detector array 40 is disposed. The scan and readout electronics may be subsequently connected with other electronics. The die or chip 60 is disposed behind the detector array 40 and the substrate 34 to minimize exposure to incident radiation (since the radiation first impacts the detector array 40 and then the substrate 34). As depicted, the die or chip 60 is coupled to the detector array 40 via metal traces 66 extending from the detector array 40 around an edge 68 of the substrate 34. In certain embodiment, the traces 66 may be additively manufactured (e.g., 3D printed) on the substrate 34.

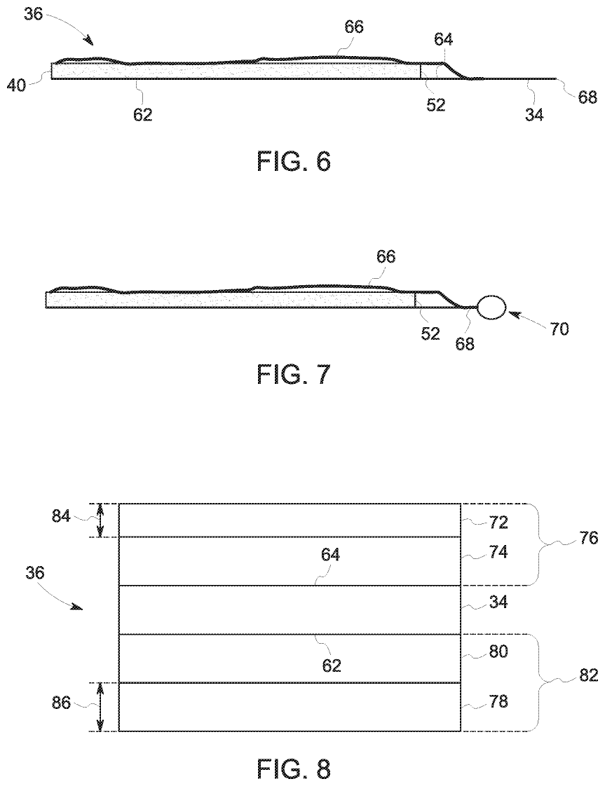

FIGS. 6 and 7 are diagrammatical representations of the detector array 40 being sealed. As depicted, the detector array 40 (e.g., including the scintillator and the imager) is disposed on the polymeric substrate 34. In certain embodiments, the scintillator may be also be flexible (e.g., flexible CsI plate or flexible GOS sheet), thus (in combination with a flexible seal), enabling the whole assembly to be flexible. A semi-hermetic coating or hermetic conductive coating is disposed on the side 52 of the substrate 34 opposite the side 64 where the detector array 40 is disposed. A seal 66 (e.g., flexible seal) is disposed over the detector array 40 to encompass the array between the substrate 34 and the seal 66. The semi-hermetic coating or hermetic conductive coating on the substrate 34 and/or the seal 66 may be a metal foil (e.g., aluminum) vacuumed sealed on, a spray coat sealant, and/or a plastic film vacuum deposited on. In certain embodiments, the coating and/or seal may act as a flexible, electromagnetic interference shield. In certain embodiments, the seal 66 may include a reflective layer configured to reflect optical photons emitted by the scintillator layer towards the imager to increase signal capture. The seal 66 is coupled (e.g., bonded) to the substrate 34 at a location past the edge 52 of the detector array 40. As depicted in FIG. 6, the seal 66 is bonded on the substrate 34 upstream of an edge 68 of the substrate 34. As depicted in FIG. 7, the seal 66 is bonded at the edge 68 of the substrate 34 and the edge 68 is treated to form an edge seal 70. The edge seal 70 may be formed via taping, painting, or coating (e.g., via an additive process). The sealing of the detector array 40 between the seal 66 and the substrate 34 forms a semi-hermetic or hermetic seal.

FIG. 8 is a diagrammatical representation of a portion of the detection subsystem 36 (e.g., configured for front side and back side irradiation at the same time) in the detector 22 of the system 10 of FIG. 1. In certain embodiments, the detector 22 may include one or more scintillators and/or one or more imagers. As depicted, a first scintillator 72 and a first imager 74 (e.g., forming a first digital detector array 76) are disposed on the side 64 of the substrate 34. The first digital detector array 76 is configured to be utilized in front side irradiation. A second scintillator 78 and a second imager 80 (e.g., forming a second digital detector array 82) are disposed on the side 62 opposite side 64 of the substrate 34. The second digital detector array 82 is configured to be utilized in back side irradiation. The substrate 34, as discussed above, may be a polymeric (e.g., flexible polyimide) substrate. In certain embodiments, the substrate 34 may be a thin glass substrate. One of the digital detector arrays 76, 82 may be directly deposed on the substrate 34, while the on the other digital detector array 76, 82 may be laminated on the substrate 34. In certain embodiments, one the detector arrays 76, 82 may be temperature pressed on the substrate 34. In certain embodiments, the scintillators 72, 78 may be flexible. As depicted, the first scintillator 72 has a length 84 (e.g., thickness or height) that is less than a length 86 (e.g., thickness or height) of the second scintillator 78. The thinner first scintillator 72 may increase the modulation transfer function, while having the additional scintillator 78 increases the detective quantum efficiency (e.g., due to capture of additional X-rays). In certain embodiments, one or more the scintillators 72, 78 may also capture optical photons.

In certain embodiments, the detector 22 may include additional layers of digital detector arrays. One digital detector array could act as an ion chamber by measuring X-ray dose. Another digital detector array may be utilized for autosensing the beginning of irradiation. The image data maybe selectively read out from the digital detector arrays.

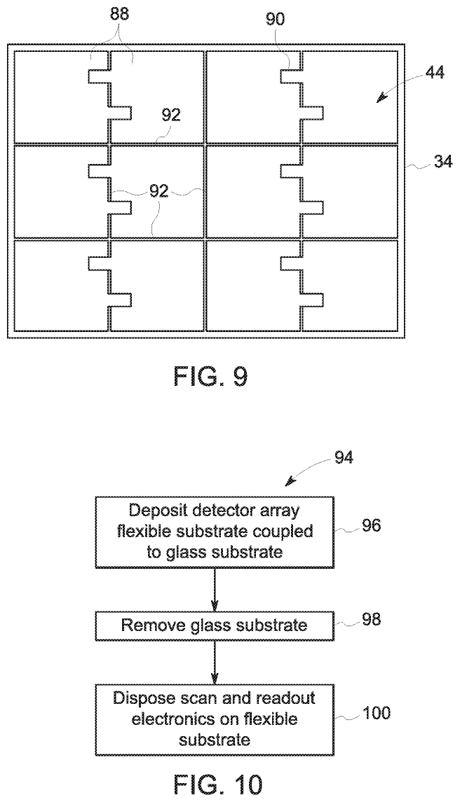

FIG. 9 is a bottom view of the imager 44 disposed on the flexible or polymeric substrate 34. As depicted, a plurality of imager modules 88 are disposed on the flexible substrate 34 (e.g., polyimide) in a tiled arrangement to form the imager 44. Each module 88 includes an extension 90 that extends behind an adjacent module 88 to enable keying of the modules 88 together. The extensions 90 along with the flexible substrate 34 enable multiplexed readout (e.g., serially) behind adjacent modules 88 (e.g., reducing the number of data modules). Tight bends within the substrate may enable the modules 88 to be close together. As depicted, multiple sides 92 of each module 88 abut multiple adjacent modules 88. In certain embodiments, one or more modules 88 abut adjacent modules 88 on each side 92 (e.g., four sides).

FIG. 10 is an embodiment of a method 94 for manufacturing the detector subsystem 36 of the detector 22 of the system 10 of FIG. 1. One or more of the steps may be performed in a different order from those depicted in the method 94. The method 94 includes depositing a detector array (e.g., scintillator and imager) on a flexible or polymeric substrate (e.g., polyimide) that is coupled to a firm substrate (e.g., glass) (block 96). In certain embodiments, the flexible substrate extends beyond the detector array. In certain embodiments, the flexible substrate has a bend as described above. The method 94 also includes removing the glass substrate from the flexible substrate (block 98). The method 94 further includes disposing scan and readout electronics on the flexible substrate. In certain embodiments, the scan and readout electronics may be disposed on the bend or after the bend in the flexible substrate. In certain embodiments, the scan and readout electronics are disposed behind the detector array and the flexible substrate.

Technical effects of the disclosed embodiments include providing a detector that includes a detection subsystem including a flexible polymeric substrate (e.g., polyimide). The flexible substrate, in conjunction with other flexible detector components (e.g., scintillator, seal, etc.), provides a more flexible detector assembly and more rugged detector assembly. In addition, the flexible substrate may enable the expansion of the detector array closer to the edges of the detector. In certain embodiments, the flexible substrate may enable fewer electronic components (e.g., data modules) and enable multiplexed readout of the image data.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A digital X-ray detector, comprising:
a detector subsystem, comprising:
a polymeric substrate;
a detector array configured to generate image data based on incident X-ray radiation disposed on the polymeric substrate, wherein the polymeric substrate extends beyond an edge of the detector array;
scan electronics and readout electronics configured to acquire the image data from the detector array, wherein the scan electronics, the readout electronics, or both the scan electronics and the readout electronics are directly disposed on the polymeric substrate; and
wherein the detector subsystem is configured for both front side irradiation and back side irradiation.

2. The digital X-ray detector of claim 1, wherein the scan electronics and the readout electronics are disposed behind the detector array relative to a side of the digital X-ray detector receiving the incident X-ray radiation to minimize exposure of the scan electronics and the readout electronics to the incident X-ray radiation.

3. (canceled)

4. (canceled)

5. The digital X-ray detector of claim 1, wherein the polymeric substrate extends in a first direction, a portion of the polymeric substrate extending beyond the edge of the detector array is bent, and the polymeric substrate extends in a second direction opposite the first direction after the bend.

6. The digital X-ray detector of claim 5, wherein the scan electronics and readout electronics are disposed on the bend or after the bend of the polymeric substrate.

7. The digital X-ray detector of claim 1, wherein the detector array comprises a scintillator disposed on the polymeric substrate configured to convert the incident radiation into lower energy optical photons.

8. The digital X-ray detector of claim 7, comprises a semi-hermetic or hermetic conductive coating disposed on a lateral side of the polymeric substrate opposite the scintillator and a flexible seal disposed over the scintillator, wherein the flexible seal is bonded to the polymeric substrate to semi-hermetically or hermetically seal the scintillator so that the edge of the polymeric substrate is also semi-hermetically or hermetically sealed

9. The digital X-ray detector of claim 8, wherein the flexible seal comprises a reflective layer configured to reflect optical photons emitted by the scintillator towards the detector array.

10. The digital X-ray detector of claim 1, wherein the polymeric substrate is transparent to enable alignment of the scan electronics and readout electronics when disposed on the polymeric substrate.

11. The digital X-ray detector of claim 1, wherein the detector array comprises a plurality of imager modules tiled together on the polymeric substrate so that at least one imager module of the plurality of imager modules abuts an adjacent imager module on each side.

12. The digital X-ray detector of claim 11, wherein each image module of the plurality of imager modules is configured for multiplexed readout behind an adjacent imager module.

13. (canceled)

14. The digital X-ray detector of claim 1, wherein the detector array comprises a first scintillator disposed on a first side of the polymeric substrate and a second scintillator disposed on a second side of polymeric substrate opposite the first side, wherein the first and second scintillators are configured to convert the incident radiation into lower energy optical photons.

15. The digital X-ray detector of claim 14, wherein the first scintillator is thinner than the second scintillator.

16. The digital X-ray detector of claim 1, wherein the detector subsystem comprises a rigid support member, and the polymeric substrate is bonded to the rigid support member.

17. A digital X-ray detector, comprising:
a detector subsystem, comprising:
a polymeric substrate;
a detector array configured to generate image data based on incident X-ray radiation disposed on the polymeric substrate, wherein the detector array comprises a scintillator configured to convert the incident radiation into lower energy optical photons, a semi-hermetic or hermetic conductive coating is disposed on a lateral side of the polymeric substrate opposite the scintillator and a flexible seal is disposed over the scintillator, wherein the flexible seal is bonded to the polymeric substrate at a location beyond an edge of the detector array to semi-hermetically or hermetically seal the scintillator so that the edge of the polymeric substrate is also semi-hermetically or hermetically sealed.

18. The digital X-ray detector of claim 17, wherein the polymeric substrate bends beyond the edge of the detector array, and the digital X-ray detector comprises scan electronics and readout electronics configured to acquire the image data from the detector array, wherein the scan electronics, readout electronics, or both the scan electronics and the readout electronics are directly disposed on the polymeric substrate at the bend or beyond the bend of the polymeric substrate.

19. The digital X-ray detector of claim 18, wherein the detector array comprises a plurality of imager modules tiled together on the polymeric substrate so that at least one imager module of the plurality of imager modules abuts an adjacent imager module on each side.

20. A method for manufacturing a digital X-ray detector, comprising:
depositing a detector array configured to generate image data based on incident X-ray radiation on a polymeric substrate disposed on a glass substrate wherein the polymeric substrate extends in a first direction beyond an edge of the detector array and then bends and extends in a second direction opposite the first direction after the bend;
removing the glass substrate from the polymeric substrate; and
directly disposing scan electronics, readout electronics, or both the scan electronics and the readout electronics on the polymeric substrate on the bend or after the bend of the polymeric substrate, wherein the scan electronics and the readout electronics are configured to acquire the image data from the detector array.
